(19) United States
(12) Patent Application Publication
(10) Pub. No.: US 2007/0185204 A1
(43) Pub. Date: Aug. 9, 2007

(54) Crystalline forms of 3-biphenyl-4-yl-(2S)-[(4'-trifluoromethyl-biphenyl-4-carbonyl)-amino]-propionic acid, and methods of use

(76) Inventors: Eric J. Benjamin, Jamestown, NC (US); Devi R. Gohimukkula, Jamestown, NC (US)

Correspondence Address: KILPATRICK STOCKTON LLP - 41305;CHARLES CALKINS, 1001 WEST FOURTH STREET, WINSTON-SALEM, NC, 27101, US

(21) Appl. No.: 11/651,208

(22) Filed: Jan. 9, 2007

Related U.S. Application Data
(60) Provisional application No. 60/758,740, filed on Jan. 13, 2006.

(51) Int. Cl. A61K 31/195; C07C 237/20

(52) U.S. Cl. 514/563; 562/450

(57) ABSTRACT

The present invention relates to crystalline Forms of 3-biphenyl-4-yl-(2S)-[(4′-trifluoromethyl-biphenyl-4-carbonyl)-amino]-propionic acid. In one embodiment, the present invention provides polymorphic Forms I, II, and III of 3-biphenyl-4-yl-(2S)-[(4′trifluoromethyl-biphenyl-4-carbonyl)-amino]-propionic acid.

The present invention claims priority under 35 U.S.C. 119(e) to U.S. Provisional Application 60/758,740 filed Jan. 13, 2006, the entire contents of which are incorporated by reference in its entirety.

### FIELD OF THE INVENTION

The present invention relates to crystalline forms of 3-biphenyl-4-yl-(2S)-[(4′-trifluoromethyl-biphenyl-4-carbonyl)-amino]-propionic acid, which is an antagonist of the intrinsic clotting pathway.

### BACKGROUND OF THE INVENTION

Numerous strategies have been developed for the treatment of thrombotic disorders. Many antithrombotic therapies are based on interference in the hemostatic system. This approach carries the inherent risk of bleeding, since the hemostatic system is no longer fully responsive to potential injury. Therefore, antithrombotic benefits are normally associated with antihemostatic risks. In attempts to improve the benefit-to-risk ratio, antithrombotic agents are continuously being developed. Various antithrombotic strategies include administering general inhibitors of thrombin formation such as heparin or vitamin K antagonists; administering specific thrombin inhibitors; administering specific factor Xa inhibitors; and administering inhibitors of platelet activation and adhesion.

Evaluation of current antithrombotic strategies in terms of antithrombotic benefits versus antihemostatic risks reveals that the benefit-to-risk ratio tends to be more favorable for strategies that interfere with one specific step rather than in a more general phase of the hemostatic system [L. A. Harker, Biomedical Progress vol 8, 1995, 17-26]. For example, the development of inhibitors specific for factor Xa is an improvement from general and specific thrombin inhibitors. But, this approach still blocks the common (intrinsic and extrinsic) pathway of thrombin generation and thereby thrombin-dependent platelet activation. Thus, a need exists for more specific antithrombotic agents that selectively inhibit one single hemostatic pathway, while leaving other pathways unaffected.

### BRIEF SUMMARY OF THE INVENTION

In one aspect, the present invention provides polymorphic forms of a compound for the treatment of thrombotic disorders and related diseases. In one embodiment, the present invention provides a first polymorph, Form I, of 3-biphenyl-4-yl-(2S)-[(4′-trifluoromethyl-biphenyl-4-carbonyl)-amino]-propionic acid having X-ray powder diffraction peaks expressed in degrees-2θ at about 18.0 and 19.6, and a differential scanning calorimetry thermogram comprising at least an exothermic peak at about 236° C. and an endothermic peak at about 294° C.

In another embodiment, the present invention provides a second polymorph, Form II, of 3-biphenyl-4-yl-(2S)-[(4′-trifluoromethyl-biphenyl-4-carbonyl)-amino]-propionic acid having an X-ray powder diffraction peak expressed in degrees-2θ at about 16.7, and a differential scanning calorimetry thermogram comprising at least an exothermic peak at about 243° C. and an endothermic peak at about 294° C.

In another embodiment, the present invention provides a third polymorph, Form III, of 3-biphenyl-4-yl-(2S)-[(4′-trifluoromethyl-biphenyl-4-carbonyl)-amino]-propionic acid having at least two X-ray powder diffraction peaks expressed in degrees-2θ selected from 15.7, 22.3 and 22.7, and a differential scanning calorimetry (DSC) thermogram comprising an endothermic peak at about 294° C.

In another aspect, the present invention provides a method for producing a polymorph of 3-biphenyl-4-yl-(2S)-[(4′-trifluoromethyl-biphenyl-4-carbonyl)-amino]-propionic acid.

In another aspect, the present invention provides pharmaceutical compositions comprising one or more polymorphic forms of 3-biphenyl-4-yl-(2S)-[(4′-trifluoromethyl-biphenyl-4-carbonyl)-amino]-propionic acid.

In another aspect, the present invention also provides methods of producing a pharmaceutical composition comprising one or more polymorphic forms of 3-biphenyl-4-yl-(2S)-[(4′-trifluoromethyl-biphenyl-4-carbonyl)-amino]-propionic acid. Embodiments of pharmaceutical compositions of the present invention may comprise therapeutically effective amounts of one or more polymorphs of 3-biphenyl-4-yl-(2S)-[(4′-trifluoromethyl-biphenyl-4-carbonyl)-amino]-propionic acid.

In another aspect, the present invention also provides methods of treating thrombotic disorders and related diseases comprising administering a pharmaceutical composition comprising one or more polymorphic forms of 3-biphenyl-4-yl-(2S)-[(4′-trifluoromethyl-biphenyl-4-carbonyl)-amino]-propionic acid to a subject in need thereof. Embodiments of the methods of treatment of the present invention may comprise administering a pharmaceutical comprising a therapeutically effective amount of one or more polymorphs of 3-biphenyl-4-yl-(2S)-[(4′-trifluoromethyl-biphenyl-4-carbonyl)-amino]-propionic acid.

These and other embodiments of the present invention are described in greater detail in the detailed description of the invention which follows.

### BRIEF DESCRIPTION OF THE DRAWINGS

FIG. 1 is a X-ray diffraction pattern of a polymorph of 3-biphenyl-4-yl-(2S)-[(4′-trifluoromethyl-biphenyl-4-carbonyl)-amino]-propionic acid according to an embodiment of the present invention.

FIG. 2 is a differential scanning calorimetry (DSC) thermogram of a polymorph of 3-biphenyl-4-yl-(2S)-[(4′-trifluoromethyl-biphenyl-4-carbonyl)-amino]-propionic acid according to an embodiment of the present invention.

FIG. 3 is an IR spectrum of a polymorph of 3-biphenyl-4-yl-(2S)-[(4′-trifluoromethyl-biphenyl-4-carbonyl)-amino]-propionic acid according to an embodiment of the present invention.

FIG. 4 is a X-ray diffraction pattern of a polymorph of 3-biphenyl-4-yl-(2S)-[(4′-trifluoromethyl-biphenyl-4-carbonyl)-amino]-propionic acid according to an embodiment of the present invention.

FIG. 5 is a DSC thermogram of a polymorph of 3-biphenyl-4-yl-(2S)-[(4′-trifluoromethyl-biphenyl-4-carbonyl)-amino]-propionic acid according to an embodiment of the present invention.

FIG. 6 is an IR spectrum of a polymorph of 3-biphenyl-4-yl-(2S)-[(4′-trifluoromethyl-biphenyl-4-carbonyl)-amino]-propionic acid according to an embodiment of the present invention.

FIG. 7 is a X-ray diffraction pattern of a polymorph of 3-biphenyl-4-yl-(2S)-[(4′-trifluoromethyl-biphenyl-4-carbonyl)-amino]-propionic acid according to an embodiment of the present invention.

FIG. 8 is a DSC thermogram of a polymorph of 3-biphenyl-4-yl-(2S)-[(4′-trifluoromethyl-biphenyl-4-carbonyl)-amino]-propionic acid according to an embodiment of the present invention.

### DETAILED DESCRIPTION

For the purposes of this specification, unless otherwise indicated, all numbers expressing quantities of ingredients, reaction conditions, and so forth used in the specification are to be understood as being modified in all instances by the term “about.” Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

By percent by weight it is meant that a particular weight of one ingredient in a composition is divided by the total weight of all of the ingredients in that composition. Percent by weight may be used interchangeably and means approximately the same as weight/weight percent or %( weight/weight) or percent by mass or mass percent. When a liquid solute is used, it is often more practical to use volume/volume percent or % (vol/vol) or percent by volume, which are all considered to be synonymous. Ppm (parts per million), ppb (parts per billion), pph (parts per hundred) are often used to indicate a percentage based on quantity and not on mass (i.e., the quantity of a given type of atom or a given type of molecule in a composition with more atoms or molecules (be it gas, liquid or solid) is divided by the total quantity of atoms or molecules in the total composition). Other terms that are used are molarity, which is the number of moles of solute per liters of solution, molality, which is the number of moles of solution per kilograms of solution. Another concentration unit is the mole fraction, which is the moles of a given component divided by the total moles of all solution components. Mole percent is related to the mole fraction and is the mole fraction multiplied by 100.

It is further noted that, as used in this specification, the singular forms “a,” “an,” and “the” include plural referents unless expressly and unequivocally limited to one referent.

The term “factor IX” is used herein to refer to blood coagulation factor IX, including both activated and non-activated forms thereof.

The term “therapeutically effective amount” is used herein to denote the amount of 3-biphenyl-4-yl-(2S)-[(4′-trifluoromethyl-biphenyl-4-carbonyl)-amino]-propionic acid that will elicit the therapeutic response of a subject that is being sought. In an embodiment, the therapeutic response may be partial inhibition of the biological function of factor IX.

In another embodiment, the therapeutically effective amount may be a sustained blood level of 3-biphenyl-4-yl-(2S)-[(4′-trifluoromethyl-biphenyl-4-carbonyl)-amino]-propionic acid of less than 1 μM. In another embodiment, the therapeutically effective amount may be a sustained blood level of 3-biphenyl-4-yl-(2S)-[(4′-trifluoromethyl-biphenyl-4-carbonyl)-amino]-propionic acid of greater than 0.1 μM. In an embodiment, such a concentration may be sustained for at least 1 hour. In another embodiment, such a concentration may be sustained for at least 2 hours. In another embodiment, such a concentration may be sustained for at least 6 hours. In another embodiment, such a concentration may be sustained for at least 8 hours. In another embodiment, such a concentration may be sustained for at least 12 hours. In another embodiment, such a concentration may be sustained for at least 24 hours.

In another embodiment, the dosage or blood level of 3-biphenyl-4-yl-(2S)-[(4′-trifluoromethyl-biphenyl-4-carbonyl)-amino]-propionic acid and administration may be sufficient for inhibition of the biological function of factor IX at a sufficient level for sufficient time to inhibit thrombosis or to induce a therapeutic effect.

A therapeutically effective amount may be achieved in a subject by administering a dosage level of less than 10 mg of compound/kg of body weight per day. In another embodiment, the dosage level of administration is greater than 0.5 mg of compound/kg of body weight per day.

The term “treatment” as used herein, refers to the full spectrum of treatments for a given condition or disorder from which a subject is suffering, including alleviation of one, most, or all of the symptoms resulting from that disorder, to the prevention of the onset of the disorder.

As used herein 3-biphenyl-4-yl-(2S)-[(4′-trifluoromethyl-biphenyl-4-carbonyl)-amino]-propionic acid refers to the composition disclosed and described in United States Patent Application No. 10/637,900 (Publication No. 2004-0110832), which is hereby incorporated by reference in its entirety.

In one aspect, the present invention provides polymorphic forms of compounds for the treatment of thrombotic disorders and related diseases. In one embodiment, the present invention provides a first polymorph, Form I, of 3-biphenyl-4-yl-(2S)-[(4′-trifluoromethyl-biphenyl-4-carbonyl)-amino]-propionic acid having X-ray powder diffraction peaks expressed in degrees-2θ at about 18.0 and 19.6, and a differential scanning calorimetry thermogram comprising at least an exothermic peak at about 236° C. and an endothermic peak at about 294° C. Form I may further have X-ray powder diffraction peaks expressed in degrees-2θ at about 20.1 and 21.1. In another embodiment, Form I may have at least three X-ray powder diffraction peaks selected from the group consisting of 18.0, 19.6, 20.1, and 21.1. Form I may further have an IR spectrum in KBr comprising at least two peaks selected from about 833 cm⁻¹, 1611 cm⁻¹, and 1745 cm⁻¹. Form I may further have a ¹³C solid state NMR spectrum comprising at least two peaks selected from 173.7, 170.7, 58.0, and 35.6 ppm.

In another embodiment, the present invention provides a second polymorph, Form II, of 3-biphenyl-4-yl-(2S)-[(4′-trifluoromethyl-biphenyl-4-carbonyl)-amino]-propionic acid having an X-ray powder diffraction peak expressed in degrees-2θ at about 16.7, and a differential scanning calorimetry thermogram comprising at least an exothermic peak at about 243° C. and an endothermic peak at about 294° C. Form II may further have one or more X-ray powder diffraction peaks expressed in degrees-2θ at about 20.0, 21.5, and 26.8. In another embodiment, Form II may have at least three X-ray powder diffraction peaks selected from the group consisting of 16.7, 20.0, 21.5, and 26.8. Form II may further have an IR spectrum in KBr comprising at least two peaks selected from about 761 cm⁻¹, 1651 cm⁻¹, and 1714 cm⁻¹ in an infrared spectrum. Form II may further have a ¹³C solid state NMR spectrum comprising at least two peaks selected from 180.0, 168.2, 55.6, and 38.4 ppm.

In another embodiment, the present invention provides a third polymorph, Form III, of 3-biphenyl-4-yl-(2S)-[(4′-trifluoromethyl-biphenyl-4-carbonyl)-amino]-propionic acid having at least two X-ray powder diffraction peaks expressed in degrees-2θ selected from 15.7, 22.3 and 22.7, and a differential scanning calorimetry thermogram comprising an endothermic peak at about 294° C. Form III may further have one or more X-ray powder diffraction peaks expressed in degrees-2θ at about 3.8, 19.4, and 21.4. In another embodiment, Form III has no exothermic peaks.

In another aspect, the present invention provides a method for producing a polymorph of 3-biphenyl-4-yl-(2S)-[(4′-trifluoromethyl-biphenyl-4-carbonyl)-amino]-propionic acid. In an embodiment, the method of producing a polymorph comprises: dissolving 3-biphenyl-4-yl-(2S)-[(4′-trifluoromethyl-biphenyl-4-carbonyl)-amino]-propionic acid in a solvent system comprising a solvent selected from the group consisting of: an alcoholic solvent, acetone, ethyl acetate, THF, HPCD, DMA, water, and mixtures thereof, and recovering the precipitate from the solvent system. In an embodiment, a polymorph produced by this method is Form I. In another embodiment, a polymorph produced by this method is Form II. In another embodiment, the alcoholic solvent is methanol. In another embodiment, the solvent system comprises an alcoholic solvent. In another embodiment, the step of dissolving comprises heating the solvent system. The solvent system may be heated to a temperature above ambient temperature up to and including refluxing temperature. In another embodiment, the step of recovering the precipitate from the solvent system comprises cooling the solvent system.

In one non-limiting embodiment, solid 3-biphenyl-4-yl-(2S)-[(4′-trifluoromethyl-biphenyl-4-carbonyl)-amino]-propionic acid may be dissolved (either partially or completely) in an alcoholic solvent such as, but not limited to, methanol by heating the mixture to a temperature above room temperature. In an embodiment where the alcoholic solvent is methanol, the mixture is heated to reflux. The polymorph is recovered from the alcoholic solvent by cooling the mixture and separating the polymorph by filtration. The polymorph may be further dried under vacuum. Form I and II have each been produced using the above method.

Form II may be produced using the above method where a refluxed solution of the compound in methanol is either cooled quickly by placing the refluxed solution directly into an ice bath or cooled slowly by allowing the refluxed solution to cool to room temperature on the bench top.

In another embodiment, the present invention provides a method for producing a polymorph of 3-biphenyl-4-yl-(2S)-[(4′-trifluoromethyl-biphenyl-4-carbonyl)-amino]-propionic acid comprising heating solid 3-biphenyl-4-yl-(2S)-[(4′-trifluoromethyl-biphenyl-4-carbonyl)-amino]-propionic acid to a temperature above an exothermic transition temperature and cooling the resulting compound to produce Form III. In an embodiment, wherein said heated solid is Form I of 3-biphenyl-4-yl-(2S)-[(4′-trifluoromethyl-biphenyl-4-carbonyl)-amino]-propionic acid, the exothermic transition temperature is about 236° C. at a ramp speed of 10° C./min. In another embodiment, wherein said heated solid is Form II of 3-biphenyl-4-yl-(2S)-[(4′-trifluoromethyl-biphenyl-4-carbonyl)-amino]-propionic acid, the exothermic transition temperature is about 243° C. at a ramp speed of 10° C./min. In another embodiment, solid 3-biphenyl-4-yl-(2S)-[(4′-trifluoromethyl-biphenyl-4-carbonyl)-amino]-propionic acid is heated to 255° C. and cooled to produce Form III.

To ensure that no chemical transformation or degradation has occurred, the purity of each polymorph may be confirmed according to HPLC and then characterized by its physico-chemical properties such as DSC, X-ray diffraction, infrared spectrum, and/or solid state ¹³C NMR.

In another aspect, the present invention provides pharmaceutical compositions comprising one or more polymorphic forms of 3-biphenyl-4-yl-(2S)-[(4′-trifluoromethyl-biphenyl-4-carbonyl)-amino]-propionic acid. In one embodiment, a pharmaceutical composition may comprise Form I of 3-biphenyl-4-yl-(2S)-[(4′-trifluoromethyl-biphenyl-4-carbonyl)-amino]-propionic acid. In another embodiment, a pharmaceutical composition may comprise Form II of 3-biphenyl-4-yl-(2S)-[(4′-trifluoromethyl-biphenyl-4-carbonyl)-amino]-propionic acid. In another embodiment, a pharmaceutical composition may comprise Form I and Form II of 3-biphenyl-4-yl-(2S)-[(4′-trifluoromethyl-biphenyl-4-carbonyl)-amino]-propionic acid. In another embodiment, a pharmaceutical composition may comprise Form III of 3-biphenyl-4-yl-(2S)-[(4′-trifluoromethyl-biphenyl-4-carbonyl)-amino]-propionic acid.

In another aspect, the present invention also provides methods of producing a pharmaceutical composition comprising Form I and/or Form II of 3-biphenyl-4-yl-(2S)-[(4′-trifluoromethyl-biphenyl-4-carbonyl)-amino]-propionic acid. In one embodiment, a method of producing a pharmaceutical composition may comprise combining Form I of 3-biphenyl-4-yl-(2S)-[(4′-trifluoromethyl-biphenyl-4-carbonyl)-amino]-propionic acid with a pharmaceutically acceptable excipient, diluent, carrier, or a mixture thereof. In another embodiment, a method for producing a pharmaceutical composition may comprise combining Form II of 3-biphenyl-4-yl-(2S)-[(4′-trifluoromethyl-biphenyl-4-carbonyl)-amino]-propionic acid with a pharmaceutically acceptable excipient, diluent, carrier, or a mixture thereof. In another embodiment, a method for producing a pharmaceutical composition may comprise combining Form I and Form II of 3-biphenyl-4-yl-(2S)-[(4′-trifluoromethyl-biphenyl-4-carbonyl)-amino]-propionic acid with a pharmaceutically acceptable excipient, diluent, carrier, or a mixture thereof. In another embodiment, a method for producing a pharmaceutical composition may comprise combining Form III with a pharmaceutically acceptable excipient, diluent, carrier, or a mixture thereof. Embodiments of the methods of producing pharmaceutical compositions may comprise combining a therapeutically effective amount of Form I, Form II, Form III, or mixtures thereof with a pharmaceutically acceptable excipient, diluent, carrier, or a mixture thereof.

Pharmaceutical compositions of the present invention comprising a Form I, Form II, Form III, or mixtures thereof of 3-biphenyl-4-yl-(2S)-[(4′-trifluoromethyl-biphenyl-4-carbonyl)-amino]-propionic acid may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations.

Tablets may contain 3-biphenyl-4-yl-(2S)-[(4′-trifluoromethyl-biphenyl-4-carbonyl)-amino]-propionic acid in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques to form osmotic therapeutic tablets for controlled release.

Formulations for oral use may also be presented as hard gelatin capsules where one or more polymorphs of 3-biphenyl-4-yl-(2S)-[(4′-trifluoromethyl-biphenyl-4-carbonyl)-amino]-propionic acid is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may comprise one or more polymorphs of 3-biphenyl-4-yl-(2S)-[(4′-trifluoromethyl-biphenyl-4-carbonyl)-amino]-propionic acid in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending Form I and/or Form II of 3 -biphenyl-4-yl-(2S)-[(4′-trifluoromethyl-biphenyl-4-carbonyl)-amino]-propionic acid in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the one or more polymorphs of 3-biphenyl-4-yl-(2S)-[(4′-trifluoromethyl-biphenyl-4-carbonyl)-amino]-propionic acid in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing one or more polymorphs of 3-biphenyl-4-yl-(2S)-[(4′-trifluoromethyl-biphenyl-4-carbonyl)-amino]-propionic acid with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing polymorphs of the present invention are contemplated. For the purpose of this application, topical applications shall include mouth washes and gargles. The polymorph compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines. Also provided by the present invention are prodrugs of the invention.

In another embodiment, the present invention also provides pharmaceutical compositions comprising a therapeutically effective amount of 3-biphenyl-4-yl-(2S)-[(4′-trifluoromethyl-biphenyl-4-carbonyl)-amino]-propionic acid wherein a therapeutically effective amount of 3-biphenyl-4-yl-(2S)-[(4′-trifluoromethyl-biphenyl-4-carbonyl)-amino]-propionic acid comprises a sufficient amount for the treatment and/or prevention of thrombotic disorders. In another embodiment, a pharmaceutical composition may comprise a therapeutically effective amount of Form I of 3-biphenyl-4-yl-(2S)-[(4′-trifluoromethyl-biphenyl-4-carbonyl)-amino]-propionic acid. In another embodiment, a pharmaceutical composition may comprise a therapeutically effective amount of Form II of 3-biphenyl-4-yl-(2S)-[(4′-trifluoromethyl-biphenyl-4-carbonyl)-amino]-propionic acid. In another embodiment, a pharmaceutical composition may comprise a therapeutically effective amount of a mixture of Form I and Form II of 3-biphenyl-4-yl-(2S)-[(4′-trifluoromethyl-biphenyl-4-carbonyl)-amino]-propionic acid. In another embodiment, a pharmaceutical composition may comprise a therapeutically effective amount of Form III of 3-biphenyl-4-yl-(2 S)-[(4′-trifluoromethyl-biphenyl-4-carbonyl)-amino]-propionic acid. In one embodiment, a therapeutically effective amount may be an amount sufficient to maintain in a subject a sustained blood level of 3-biphenyl-4-yl-(2S)-[(4′-trifluoromethyl-biphenyl-4-carbonyl)-amino]-propionic acid of greater than 0.1 μM. In another embodiment, a therapeutically effective amount may be an amount sufficient to maintain in a subject a sustained blood level of 3-biphenyl-4-yl-(2S)-[(4′-trifluoromethyl-biphenyl-4-carbonyl)-amino]-propionic acid of less than 1.0 μM.

In another embodiment, the present invention provides a pharmaceutical composition comprising a therapeutically effective amount of Form I, Form II, Form III, or mixtures thereof of 3-biphenyl-4-yl-(2S)-[(4′-trifluoromethyl-biphenyl-4-carbonyl)-amino]-propionic acid, and a pharmaceutically acceptable carrier, excipient, diluent, or mixture thereof, and further comprises one or more therapeutic agents.

In another aspect, the present invention additionally provides methods for treating thrombotic disorders and related diseases. In one embodiment, a method for treating thrombotic disorders may comprise administering a pharmaceutical composition comprising Form I of 3-biphenyl-4-yl-(2S)-[(4′-trifluoromethyl-biphenyl-4-carbonyl)-amino]-propionic acid to a subject in need thereof. In another embodiment, a method for treating thrombotic disorders may comprise administering a pharmaceutical composition comprising Form II of 3-biphenyl-4-yl-(2S)-[(4′-trifluoromethyl-biphenyl-4-carbonyl)-amino]-propionic acid to a subject in need thereof. In another embodiment, a method for treating thrombotic disorders may comprise administering a pharmaceutical composition comprising Form III of 3-biphenyl-4-yl-(2S)-[(4′-trifluoromethyl-biphenyl-4-carbonyl)-amino]-propionic acid to a subject in need thereof. In another embodiment, a method for treating thrombotic disorders may comprise administering a pharmaceutical composition comprising Form I and Form II of 3-biphenyl-4-yl-(2S)-[(4′-trifluoromethyl-biphenyl-4-carbonyl)-amino]-propionic acid to a subject in need thereof. Form I and Form II, for example, may be administered as a mixture.

In another embodiment, a method for treating thrombotic disorders may further comprise administering a pharmaceutical composition comprising a therapeutically effective amount of 3-biphenyl-4-yl-(2S)-[(4′-trifluoromethyl-biphenyl-4-carbonyl)-amino]-propionic acid to a subject in need thereof wherein a therapeutically effective amount is an amount sufficient to treat and/or prevent thrombotic disorders. In one embodiment, a therapeutically effective amount may be an amount sufficient to maintain in a subject a sustained blood level of 3-biphenyl-4-yl-(2S)-[(4′-trifluoromethyl-biphenyl-4-carbonyl)-amino]-propionic acid of greater than 0.1 μM. In another embodiment, a therapeutically effective amount may be an amount sufficient to maintain in a subject a sustained blood level of 3-biphenyl-4-yl-(2S)-[(4′-trifluoromethyl-biphenyl-4-carbonyl)-amino]-propionic acid of less than 1.0 μM.

A pharmaceutical composition of the present invention may be administered at a dosage level of less than 10 mg of compound/kg of body weight per day. In another embodiment, the dosage level of administration is greater than 0.5 mg of compound/kg of body weight per day. The amount of active ingredient that may be combined with the carrier materials to produce a single dosage will vary depending upon the host treated and the particular mode of administration. For example, in one non-limiting embodiment, a dosage unit forms, such as a tablet, intended for oral administration to humans may contain about 100 mg of 3-biphenyl-4-yl-(2S)-[(4′-trifluoromethyl-biphenyl-4-carbonyl)-amino]-propionic acid with an appropriate and convenient amount of carrier material which may vary up to about 20 percent of the total composition. The dosage may be individualized by the clinician based on the specific clinical condition of the subject being treated. Thus, it will be understood that the specific dosage level for any particular subject will depend upon a variety of factors including the activity of the specific compound employed, the age, body weight, general health, sex, diet, time of administration, route of administration, rate of excretion, drug combination and the severity of the particular disease undergoing therapy.

### EXAMPLES

3-Biphenyl-4-yl-(2S)-[(4′-trifluoromethyl-biphenyl-4-carbonyl)-amino]-propionic acid can be prepared according to procedures A and B.

Procedure A

(2S)-Amino-3-biphenyl-4-yl-propionic acid methyl ester (1.0-1.5 eq.) is reacted with 4-bromo-benzoic acid (1.0-1.5 eq.) in a solution of dimethylformamide (DMF), o-benzotriazolyl-N,N,N′,N′-tetramethyluronium hexafluorophosphate (HBTU) (1.0-1.5 eq.), and diisopropylethylamine (DIEA) (2.0-3.0 eq.). After completion of the reaction, water is added to the solution, and the resulting mixture is extracted with ethyl acetate. The combined organic layers are washed with water and brine and are subsequently dried over sodium sulfate. The solvent is removed in vacuo to afford 3-biphenyl-4-yl-(2S)-[(5-bromo-benzoyl)-amino]-propionic acid methyl ester. The 3-biphenyl-4-yl-(2S)-[(5-bromo-benzoyl)-amino]-propionic acid methyl ester can be purified by flash chromatography if desired.

3-biphenyl-4-yl-(2S)-[(5-bromo-benzoyl)-amino]-propionic acid methyl ester (1.0 eq.) is reacted with 4-trifluoromethyl phenyl boronic acid (3.0 eq.) in 1,2 dimethoxyethane (DME) or toluene using palladium tetrakis-triphenylphosphine (Pd(PPh₃)₄) (0.05 eq.), 2N Na₂CO₃ solution. The mixture was heated at 75° C. for 12 h. After completion of the reaction, solvent was evaporated in vacuo. During the reaction, some of the methyl ester may be hydrolyzed to the corresponding acid. As a result, the crude reaction product may be re-esterified by dissolving it in methanol containing 1% HCl and refluxing. After completion of the reaction, the mixture is concentrated in vacuo and the residue is purified by column chromatography to provide 3-biphenyl-4-yl-(2S)-[(4′-trifluoromethyl-biphenyl-4-carbonyl)-amino]-propionic methyl ester.

The 3-biphenyl-4-yl-(2S)-[(4′-trifluoromethyl-biphenyl-4-carbonyl)-amino]-propionic methyl ester is hydrolyzed in tetrahydrofuran/methanol (4:1) and 2N-lithium hydroxide solution (5 eq.) is added. The mixture is stirred at 0° C. and is subsequently warmed to room temperature. After completion of the reaction, 2N HCl is added to acidify the mixture, and the mixture is extracted with ethyl acetate. The organic layer is washed with brine and is dried over sodium sulfate. The solvent is removed in vacuo to afford solid 3-biphenyl-4-yl-(2S)-[(4′-trifluoromethyl-biphenyl-4-carbonyl)-amino]-propionic acid.

Procedure B

Step A—4′-trifluoromethyl-biphenyl-4-carboxylic acid

A solution is prepared by mixing water and acetonitrile (1:1), Na₂CO₃ (2.0 eq.), and carboxybenzene boronic acid (1.0 eq.). 4-bromobenzotrifluoride (1.2 eq.) and Pd(PPh₃)₄ (0.05 eq.) are added to the prepared solution. The solution is heated to about 76° C. until the reaction is complete. Completion of the reaction may be determined by HPLC.

The reaction mixture is subsequently cooled and filtered through a celite pad. The celite pad is washed successively with a solution of water/acetonitrile (1:1). The resulting filtrate is cooled in an ice bath, and 3N HCl was slowly added to the filtrate until the filtrate achieves a pH of about 2. A precipitate of 4′-trifluoromethyl-biphenyl-4-carboxylic acid forms and is separated from the solution by filtration. The 4′-trifluoromethyl-biphenyl-4-carboxylic acid is washed successively with water and diethyl ether. The 4′-trifluoromethyl-biphenyl-4-carboxylic acid is dried in vacuo at 45° C. and is not subjected to further purification.

Step B—4,4′-Biphenylalanine methyl ester hydrochloride

Thionyl chloride (SOCl₂) (1.5 eq.) is slowly added to a solution of L-4,4′-biphenylalanine (1.0 eq.) in anhydrous methanol. The resulting mixture is refluxed until the reaction is complete. Completion of the reaction may be determined by HPLC.

After completion, the reaction mixture is concentrated in vacuo using a water bath (45-50° C.) forming a solid residue. The solid residue is taken up in methanol/toluene (1:1) and is concentrated in vacuo using a water bath (45-50° C.). The resulting 4,4′-biphenylalanine methyl ester is dried in vacuo and is not further purified.

Step C—3-Biphenyl-4-yl-(2S)-[(4′-trifluoromethyl-biphenyl-4-carbonyl-amino]-propionic acid methyl ester

SOCl₂ (1.5 eq.) is slowly added to a solution of 4′-trifluoromethyl-biphenyl-4-carboxylic acid (1.0 eq.) in methylene chloride/DMF (20:1) at room temperature. The resulting solution is refluxed until the reaction is complete. Completion of the reaction may be determined by ¹H NMR. After completion, the reaction mixture is filtered, and the filtrate is concentrated in vacuo using a water bath (45-50° C.). The resulting 4′-trifluoromethyl-biphenyl-4-carboxylic acid chloride is taken up in toluene and the mixture is concentrated to dryness in vacuo using a water bath (45-50° C.).

To an ambient solution of 4′-trifluoromethyl-biphenyl-4-carboxylic acid chloride (1.0 eq.) and 4,4′-biphenylalanine methyl ester hydrochloride (1.1 eq.) in 1,4-dioxane is slowly added DIEA (2.25 eq.). After the addition, the reaction is followed by HPLC until complete. After completion of the reaction, the mixture is neutralized by the addition of 2N HCl and is concentrated in vacuo using a water bath (45-50° C.) until a precipitate forms. Cold water (ca. 5° C.) is added to the mixture, and the mixture is filtered. The collected residue is washed with cold water, air dried, washed with methanol, and air dried. The resulting 3-biphenyl-4-yl-(2S)-[(4′-trifluoromethyl-biphenyl-4-carbonyl)-amino]-propionic acid methyl ester is dried in vacuo at 45° C. without further purification.

Step D—3 -Biphenyl-4-yl-(2 S)- [(4′-trifluoromethyl-biphenyl-4-carbonyl)-amino]-propionic acid

To a solution of 3-biphenyl-4-yl-(2S)-[(4′-trifluoromethyl-biphenyl-4-carbonyl)-amino]-propionic acid methyl ester (1.0 eq.) is in DMF/THF (1:4) is slowly added aqueous LiOH (5.0 eq.). The reaction is followed by HPLC to determine completion. After reaction completion, the reaction mixture is cooled, and 5 M HCl is added until the mixture achieves a pH of about 2. The mixture is then concentrated in vacuo to remove the organic solvents. The concentrated mixture was diluted with water and a precipitate is formed by cooling the mixture in an ice bath. The precipitate is filtered and washed with water until the washings are neutral. The resulting 3 -biphenyl-4-yl-(2 S)-[(4′-trifluoromethyl-biphenyl-4-carbonyl)-amino]-propionic acid is air dried and then dried under vacuum.

Analytical Methods

The XRPD analysis was performed using a Shimadzu XRD-6000 X-ray powder diffractometer using Cu Kα radiation. The instrument was equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A theta-two theta continuous scan at 3° /min (0.4 sec/0.02° step) from 2.5 to 40°2θ was used. A silicon standard was analyzed to check the instrument alignment. Data were collected and analyzed using XRD-6000 v. 4.1.

DSC was performed using a TA Instruments 2920 differential scanning calorimeter. The sample was placed into an aluminum DSC pan, and the weight accurately recorded. The pan was covered with a lid, and then crimped. The sample cell was equilibrated at 25° C. and heated under a nitrogen purge at a rate of 10° C./min, up to a final temperature of 350° C. Indium metal was used as the calibration standard. Reported temperatures are at the transition maxima. DSC samples analyzed using a ramp speed of 20° C./min or 40° C./min showed shifts in the transition maxima toward higher temperatures.

FTIR spectra were obtained using a Biorad FTS-155 instrument. Samples were prepared using an agar mortar and pestle and a KBr die and pellet press.

The solid-state ¹³C cross polarization magic angle spinning (CP/MAS) NMR spectrum was acquired at ambient temperature on a Varian ^{Unity}INOVA-400 spectrometer (Larmor frequencies: ¹³C=100.542 MHz, ¹H=399.799 MHz). The sample was packed into a 4 mm PENCIL type zirconia rotor and rotated at 12 kHz at the magic angle. The spectrum was acquired with phase modulation (SPINAL-64) high power ¹H decoupling during the acquisition time using ¹H pulse width of 2.2 microseconds (90°), a ramped amplitude cross polarization contact time of 1 ms, a 30 ms acquisition time, a 10 second delay between scans, a spectral width of 45 kHz with 2700 data points, and 200 co-added scans. The free induction decay (FID) was processed using Varian VNMR 6.1 C software with 32768 points and an exponential line broadening factor of 50 Hz to improve the signal-to-noise ratio. The first three data points of the FID were back predicted using VNMR linear prediction algorithm to produce a flat baseline. The chemical shifts of the spectral peaks were externally referenced to the carbonyl carbon resonance of glycine at 176.5 ppm.

### Example 1

### Preparation and Characterization of Form I

Solid 3-biphenyl-4-yl-(2S)-[(4′-trifluoromethyl-biphenyl-4-carbonyl)-amino]-propionic acid (about 1.5 kg) was transferred to a 22 L flask with an overhead stirrer and reflux condenser. 12 L of methanol was added to the 22 L flask. The resulting solution was heated and refluxed for 1 hour. The solution was cooled in an ice water bath for 1 hour to precipitate Form I of 3-biphenyl-4-yl-(2S)-[(4′-trifluoromethyl-biphenyl-4-carbonyl)-amino]-propionic acid product. Form I was separated from the methanol by filtration and allowed to dry under vacuum for 1 hour. Form I was transferred to a drying tray and dried under vacuum at 45° C. until a constant weight was achieved. Form I was collected with a purity of 99% as determined by HPLC at 215 nm.

Form I was subsequently characterized by X-ray powder diffraction, differential scanning calorimetry (DSC), infrared spectroscopy, and solid state ¹³C NMR.

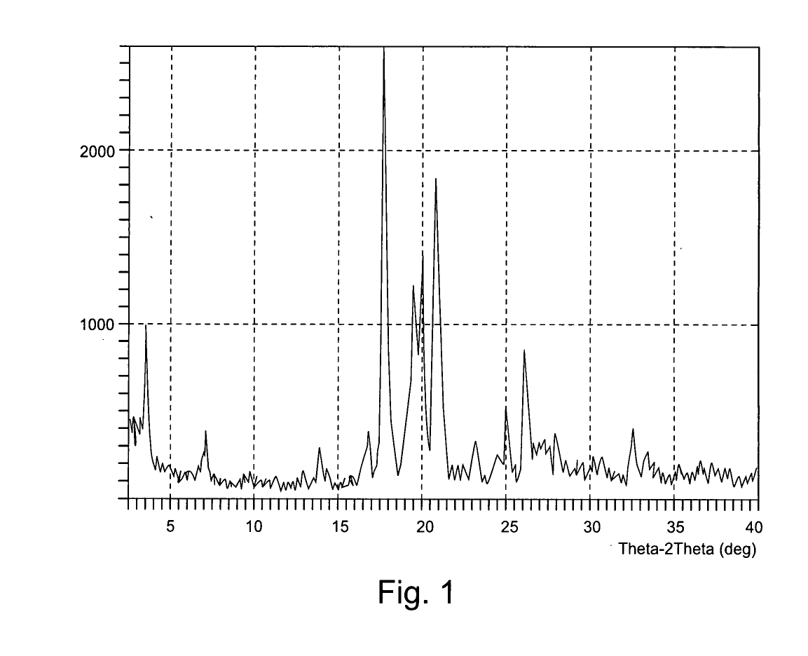

Form I displays X-ray powder diffraction peaks expressed in degrees-2θ at about 18.0, 19.6, 20.1, and 21.1, and others, as shown in FIG. 1. A complete listing of the X-ray powder diffraction peaks displayed by Form I as shown in FIG. 1 is provided in Table I.

TABLE I
XRPD of Form I

| Peak No. | Degrees-2θ | d (Angstroms) | I/Iₒ |
|---|---|---|---|
| 1 | 3.56 | 24.81 | 24 |
| 2 | 7.12 | 12.40 | 11 |
| 3 | 13.97 | 6.33 | 9 |
| 4 | 16.88 | 5.25 | 11 |
| 5 | 17.52 | 5.06 | 9 |
| 6 | 18.01 | 4.92 | 100 |
| 7 | 18.40 | 4.82 | 4 |
| 8 | 19.64 | 4.52 | 46 |
| 9 | 20.06 | 4.42 | 53 |
| 10 | 21.09 | 4.21 | 69 |
| 11 | 21.50 | 4.13 | 11 |
| 12 | 23.34 | 3.81 | 9 |
| 13 | 24.68 | 3.60 | 7 |
| 14 | 25.15 | 3.58 | 16 |
| 15 | 26.36 | 3.38 | 28 |
| 16 | 27.25 | 3.27 | 7 |
| 17 | 28.18 | 3.16 | 8 |
| 18 | 30.29 | 2.95 | 3 |
| 19 | 30.81 | 2.90 | 4 |
| 20 | 32.57 | 2.75 | 11 |
| 21 | 33.45 | 2.68 | 6 |
| 22 | 35.50 | 2.53 | 4 |
| 23 | 36.68 | 2.45 | 4 |
| 24 | 38.37 | 2.34 | 3 |
| 25 | 39.98 | 2.25 | 4 |

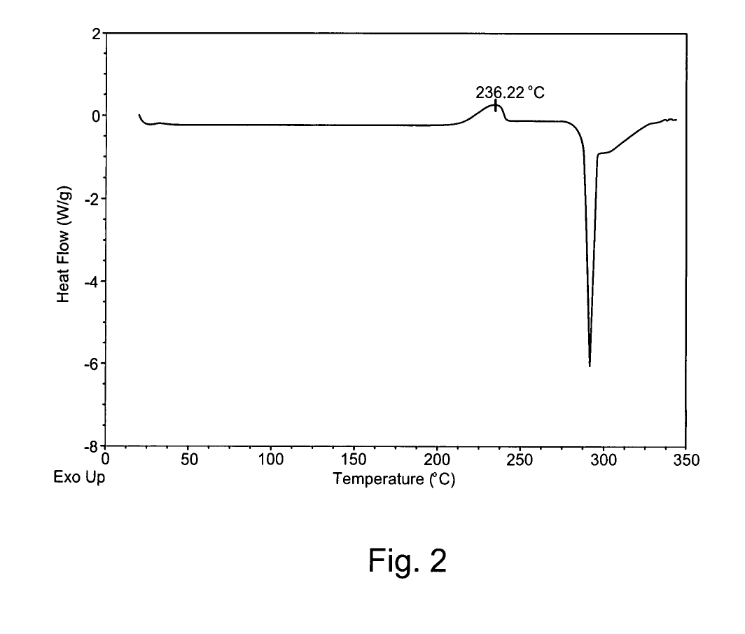

At a ramp speed of 10° C./min, the DSC profile of Form I displays an exothermic peak at about 236° C. as shown in FIG. 2. The exothermic peak at about 236° C. may be attributable to a change in crystalline forms. Form I additionally rates a melting peak at about 294° C.

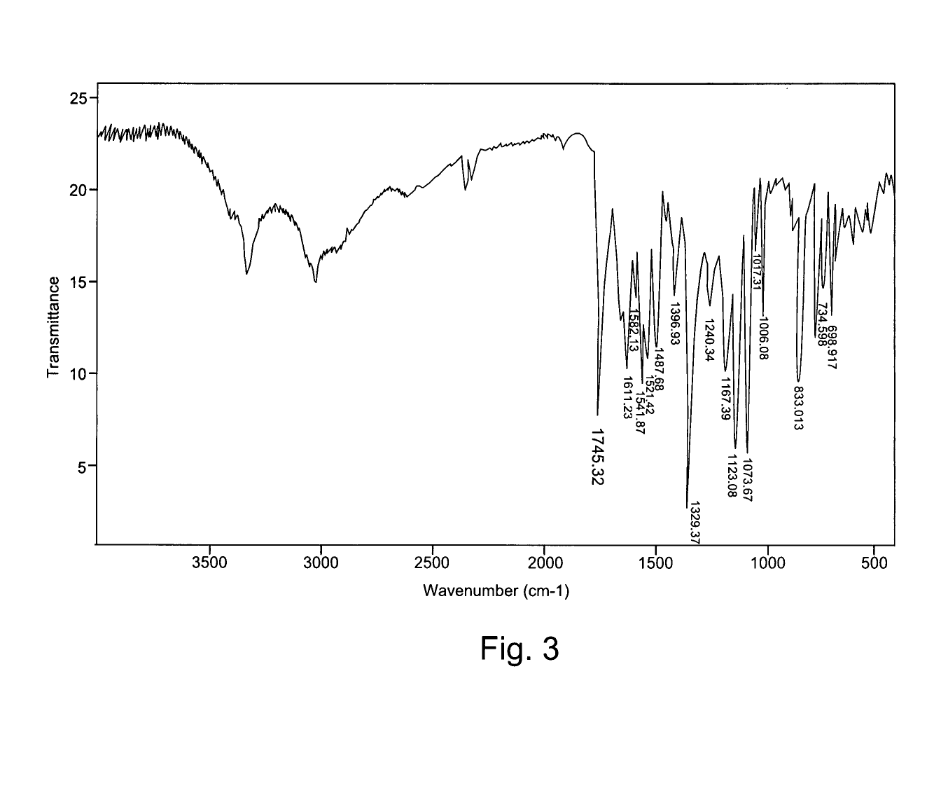

The infrared spectrum of Form I in KBr includes peaks at about 1745 cm₋₁, 1611 cm⁻¹, and 833 cm⁻¹. FIG. 3 provides a complete infrared spectrum of Form I. The peak at 1745 cm⁻¹ can be attributed to carbonyl stretching vibrations while the peak at about 1611 cm⁻¹ can be attributed to carbon double bond stretching associated with the aromatic rings of 3-biphenyl-4-yl-(2S)-[(4′-trifluoromethyl-biphenyl-4-carbonyl)-amino]-propionic acid. The peak at about 833 cm⁻¹ can be attributed to carbon single bond stretching. Additional peaks in the infrared spectrum may be used to characterize Form I.

The solid state ¹³C NMR of Form I includes peaks at about 173.7 and 170.7 ppm attributed to the two carbonyl carbons. The solid state ¹³C NMR of Form I also includes peaks at about 58.0 and 35.6 ppm attributed to the CH₂ and the CH carbons.

### Example 2

### Preparation and Characterization of Form II

Form II was prepared using the same procedure described in Example 1.

Form II was also prepared by refluxing a 1.0 g sample under the following conditions:

| | Vol. | |
|---|---|---|
| Solvent | (mL) | Crystallization Conditions |
| Methanol | 15 | Remove from heat and place |
| | | in ice bath for 1 hr. |
| Methanol | 15 | Remove from heat and allow to cool |
| | | at room temp. for 2 hrs. |
| Methanol | 30 | Remove from heat and place |
| | | in ice bath for 1 hour |
| Methanol | 30 | Remove from heat and allow |
| | | to cool at room temp. for 2 hrs. |
| Acetone | 15 | Remove from heat and allow |
| | | to cool at room temp. for 2 hrs. |
| Ethyl | 15 | Remove from heat and allow to |
| Acetate | | cool at room temp. for 2 hrs. |
| Water | 15 | Remove from heat and allow |
| | | to cool at room temp. for 2 hrs. |

After cooling, contents were filtered to remove solvent. The crystals were collected and dried for 15 minutes under vacuum. The crystals were transferred to a drying tray and dried in an oven for 1 hour at 45° C.

Form II was also prepared by the dissolving 1 gram of the solid in a mixture of THF (29 mL), MeOH (7.1 mL) and 5.14 mL of 2N LiOH was added. The solution was acidified with 2.0 N HCl to pH 3.0, and then 45 mL of ethyl acetate was added followed by 50 mL of water. The organic layer was separated and evaporated to provide Form II.

Form II was also prepared by preparing a suspension of 20.0 mg/mL of 3-biphenyl-4-yl-(2S)-[(4′-trifluoromethyl-biphenyl-4-carbonyl)-amino]-propionic acid in 5% Tween 80, 10% hydroxypropyl-b-cyclodextrin (HPCD)in deionized water. The suspension was filtered and the solid was washed with water to remove tween 80 and HPCD, and then oven dried to provide Form II.

Form II was also prepared by preparing a suspension of 20.0 mg/mL of 3-biphenyl-4-yl-(2 S)- [(4′-trifluoromethyl-biphenyl-4-carbonyl)-amino]-propionic acid in 5% Tween 80, 10% HPCD in D.I. water. The suspension was acidified with 2.0 N HCl to pH=1.0, and filtered. The solid was washed with water to remove tween 80 and HPCD, and then oven dried to provide Form II.

Form II was also prepared by preparing a suspension of 10.0 mg/mL of 3-biphenyl-4-yl-(2S)-[(4′-trifluoromethyl-biphenyl-4-carbonyl)-amino]-propionic acid in 10%, N,N-dimethyl acetamide 10%, and Tween 80 10% in Glycine buffer (pH=10). The suspension was acidified with 2.0 N HCl to pH=1.0. The resulting solid was filtered and washed with water to remove tween 80 and DMA, and then oven dried to provide Form II.

Form II was subsequently characterized by X-ray powder diffraction, differential scanning calorimetry (DSC), infrared spectroscopy, and solid state ¹³C NMR.

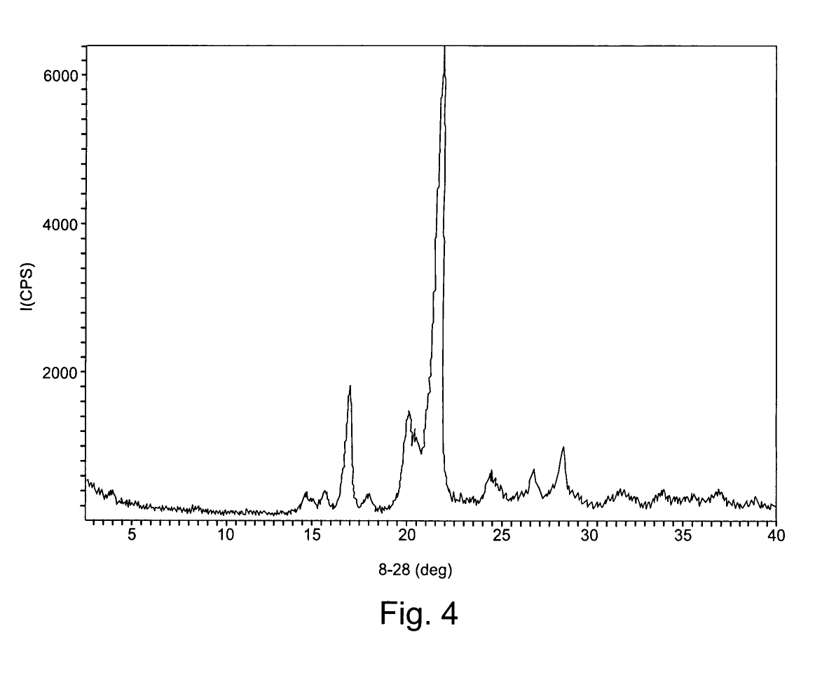

Form II displays X-ray powder diffraction peaks expressed in degrees-2θ at about 16.7, 20.0, 21.5, and 26.8, and others, as shown in FIG. 4. A complete listing of the X-ray powder diffraction peaks displayed by Form II as shown in FIG. 4 is provided in Table II.

TABLE II
XRPD of Form II

| Peak No. | Degrees-2θ | d (Angstroms) | I/Iₒ |
|---|---|---|---|
| 1 | 14.56 | 6.08 | 3 |
| 2 | 15.46 | 5.73 | 4 |
| 3 | 16.70 | 5.30 | 27 |
| 4 | 17.84 | 4.97 | 3 |
| 5 | 20.00 | 4.44 | 20 |
| 6 | 20.40 | 4.35 | 15 |
| 7 | 21.51 | 4.13 | 100 |
| 8 | 24.54 | 3.63 | 5 |
| 9 | 26.81 | 3.32 | 6 |
| 10 | 27.78 | 3.21 | 3 |
| 11 | 28.34 | 3.15 | 11 |

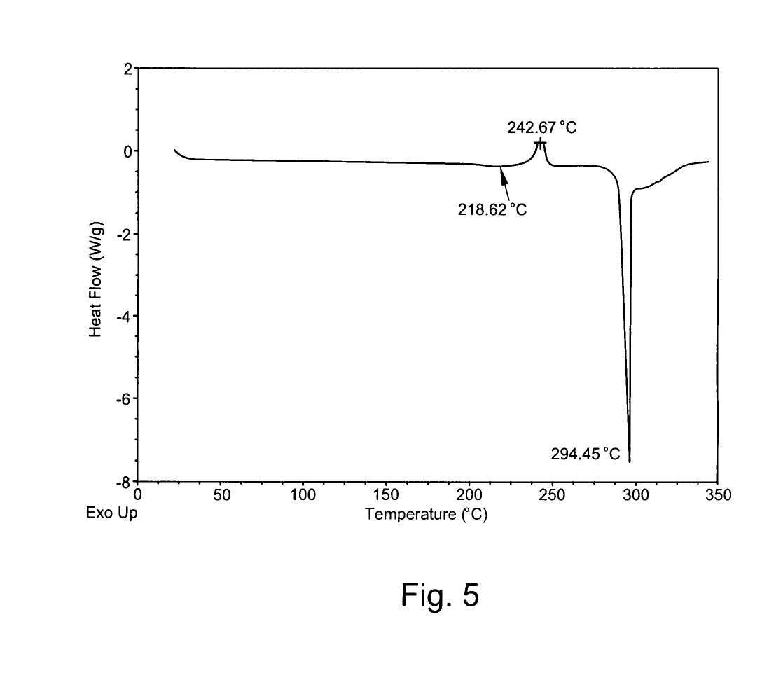

At a ramp speed of 10° C./min, the DSC profile of Form II displays an endothermic peak at about 218° C. and an exothermic peak at about 243° C. as shown in FIG. 5. These peaks may be attributable to a melting-recrystallization or a change in crystalline structure of Form II. Form II additionally displays a melting peak at about 294° C.

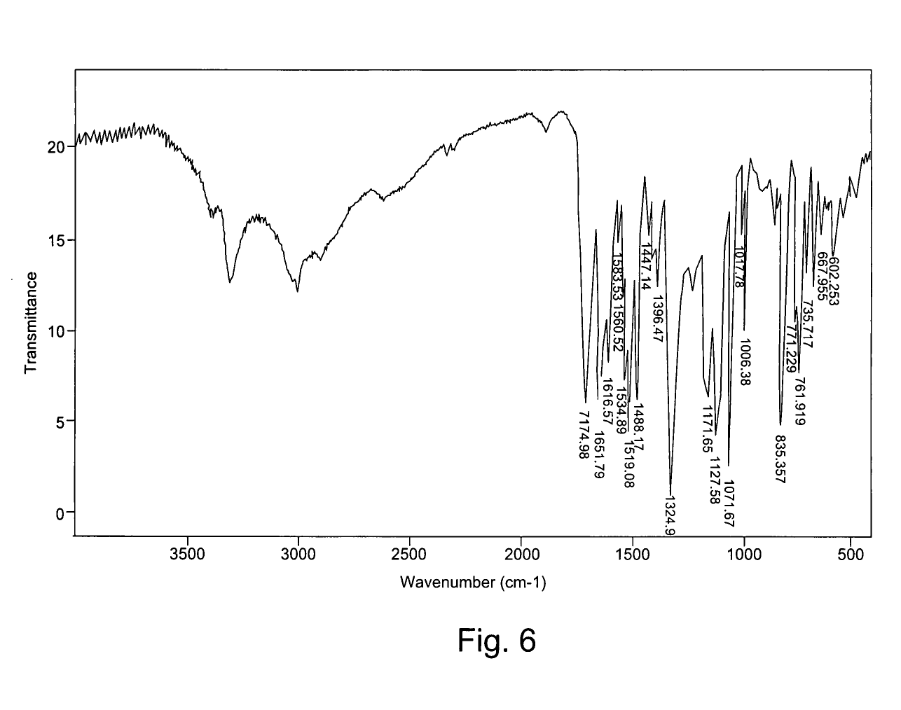

The infrared spectrum of Form II in KBr includes peaks at about 1714 cm⁻¹, 1651 cm⁻¹, and 761 cm⁻¹. FIG. 6 provides a complete infrared spectrum of Form II. The peak at 1714 cm⁻¹ can be attributed to carbonyl stretching vibrations while the peak at 1651 cm⁻¹ can be attributed to carbon double bond stretching associated with the aromatic rings of 3-biphenyl-4-yl-(2S)-[(4′-trifluoromethyl-biphenyl-4-carbonyl)-amino]-propionic acid. The peak at about 761 cm⁻¹ can be attributed to carbon single bond stretching. Additional peaks in the infrared spectrum may be used to characterize Form II.

The solid state ³C NMR of Form II includes peaks at about 180.0 and 168.2 ppm attributed to the two carbonyl carbons. The solid state ¹³C NMR of Form II also includes peaks at about 55.6 and 38.4 ppm attributed to the CH₂ and the CH carbons.

### Example 3

### Preparation and Characterization of Form III

Solid 3-biphenyl-4-yl-(2S)-[(4′-trifluoromethyl-biphenyl-4-carbonyl)-amino]-propionic acid was heated above a first exothermic transition temperature to 255° C., and then cooled to room temperature to provide Form III.

Form III was subsequently characterized by X-ray powder diffraction and differential scanning calorimetry (DSC).

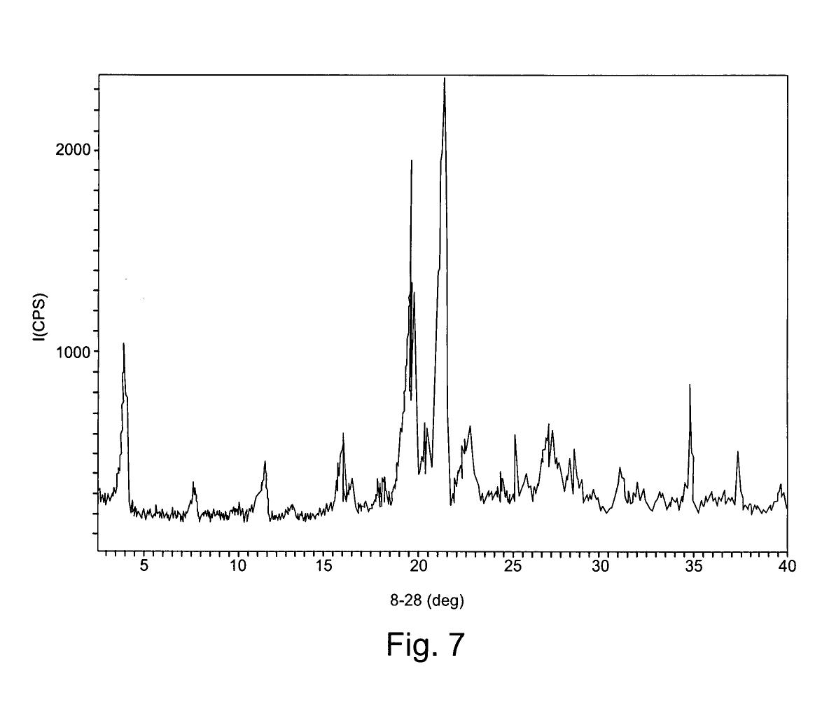

Form III displays X-ray powder diffraction peaks expressed in degrees-2θ at about 15.7, 19.4, 21.2, 22.3, 22.7, and 27.0, and others, as shown in FIG. 7. A complete listing of the X-ray powder diffraction peaks displayed by Form III as shown in FIG. 7 is provided in Table III.

TABLE III
XRPD of Form III

| Peak No. | Degrees-2θ | d (Angstroms) | I/Iₒ |
|---|---|---|---|
| 1 | 3.87 | 22.80 | 35 |
| 2 | 7.66 | 11.53 | 6 |
| 3 | 11.10 | 7.96 | 5 |
| 4 | 11.42 | 7.75 | 11 |
| 5 | 12.98 | 6.82 | 3 |
| 6 | 15.72 | 5.63 | 14 |
| 7 | 16.22 | 5.46 | 5 |
| 8 | 17.89 | 4.95 | 4 |
| 9 | 18.78 | 4.72 | 10 |
| 10 | 19.41 | 4.57 | 51 |
| 11 | 20.28 | 4.38 | 15 |
| 12 | 21.17 | 4.19 | 100 |
| 13 | 22.34 | 3.98 | 12 |
| 14 | 22.72 | 3.91 | 15 |
| 15 | 24.4 | 3.65 | 4 |
| 16 | 25.17 | 3.54 | 9 |
| 17 | 25.74 | 3.46 | 5 |
| 18 | 26.40 | 3.73 | 4 |
| 19 | 26.97 | 3.30 | 18 |
| 20 | 27.52 | 3.24 | 9 |
| 21 | 28.28 | 3.15 | 12 |
| 22 | 28.78 | 3.10 | 4 |
| 23 | 29.35 | 3.04 | 3 |
| 24 | 30.86 | 2.89 | 9 |
| 25 | 31.87 | 2.80 | 4 |
| 26 | 33.01 | 2.71 | 3 |
| 27 | 34.74 | 2.58 | 22 |
| 28 | 37.38 | 2.40 | 8 |
| 29 | 39.61 | 2.27 | 4 |

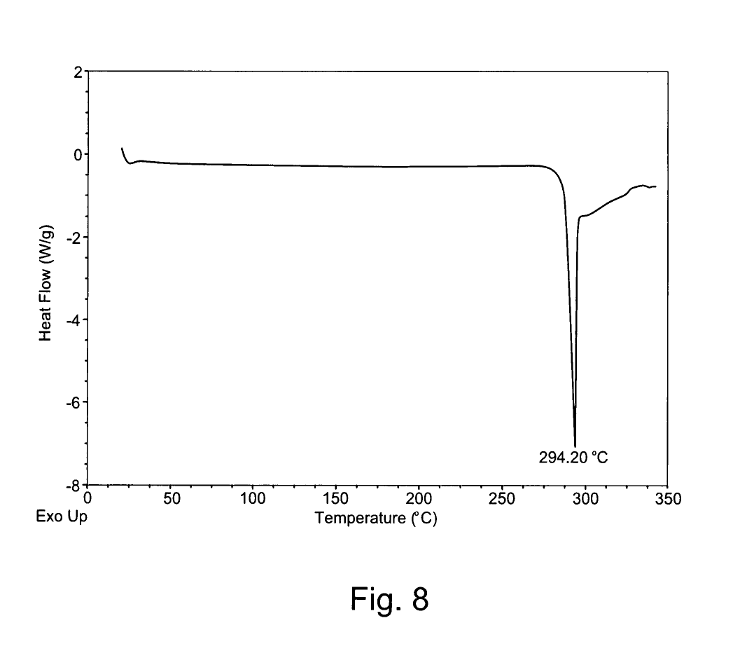

At a ramp speed of 10° C./min, the DSC profile of Form III displays an endothermic melting peak at about 294° C. as shown in FIG. 8.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the present invention.

That which is claimed is:

1. A polymorph of 3-biphenyl-4-yl-(2S)-[(4′-trifluoromethyl-biphenyl-4-carbonyl)-amino]-propionic acid, Form I, having the following characteristics:
(a) X-ray powder diffractions peaks expressed in degrees-2θ at about 18.0, and 19.6; and
(b) a differential scanning calorimetry thermogram comprising at least an exothermic peak at about 236° C. and an endothermic peak at about 294° C.

2. The polymorph of claim 1, further having X-ray powder diffraction peaks expressed in degrees-2θ at about 20.1 and 21.1.

3. The polymorph of claim 1, further having an IR spectrum in KBr comprising at least two peaks selected from 833 cm⁻¹, 1611 cm⁻¹, and 1745 cm⁻¹.

4. The polymorph of claim 1, further having a solid state ¹³C NMR spectrum comprising at least two peaks selected from 173.7, 170.7, 58.0 and 35.6 ppm.

5. A therapeutically effective amount of the polymorph of claim 1.

6. A polymorph of 3-biphenyl-4-yl-(2S)-[(4′-trifluoromethyl-biphenyl-4-carbonyl)-amino]-propionic acid, Form I, having the following characteristics:
(a) having at least three X-ray powder diffraction peaks expressed in degrees-2θ selected from the group consisting of: 18.0, 19.6, 20.1, 21.1; and
(b) having solid state ¹³C NMR spectrum comprising at least two peaks selected from 173.7, 170.7, 58.0 and 35.6 ppm.

7. The polymorph of claim 6, further having an IR spectrum in KBr comprising at least two peaks selected from 833 cm⁻¹, 1611 cm⁻¹, and 1745 cm⁻¹.

8. A therapeutically effective amount of the polymorph of claim 6.

9. A polymorph of 3-biphenyl-4-yl-(2S)-[(4′-trifluoromethyl-biphenyl-4-carbonyl)-amino]-propionic acid, Form II, having the following characteristics:
(a) an X-ray powder diffraction peak expressed in degrees-2θ at about 16.7; and
(b) a differential scanning calorimetry thermogram comprising at least an exothermic peak at about 243° C. and an endothermic peak at about 294° C.

10. The polymorph of claim 9, further having X-ray powder diffraction peaks expressed in degrees-2θ at about 20.0, 21.5, and 26.8.

11. The polymorph of claim 9, further having an IR spectrum in KBr comprising at least two peaks selected from 761 cm⁻¹, 1651 cm⁻¹, and 1714 cm⁻¹.

12. The polymorph of claim 9, further having solid state ¹³C NMR spectrum comprising at least two peaks selected from 180.0, 168.2, 55.6, and 38.4 ppm.

13. A therapeutically effective amount of the polymorph of claim 9.

14. A polymorph of 3-biphenyl-4-yl-(2S)-[(4′-trifluoromethyl-biphenyl-4-carbonyl)-amino]-propionic acid, Form II, having the following characteristics:
(a) having at least three X-ray powder diffraction peaks expressed in degrees-2θ selected from the group consisting of: 16.7, 20.0, 21.5, and 26.8; and
(b) having solid state ¹³C NMR spectrum comprising at least two peaks selected from 180.0, 168.2, 55.6, and 38.4 ppm.

15. The polymorph of claim 14,-further having an IR spectrum in KBr comprising at least two peaks selected from 761 cm⁻¹, 1651 cm⁻¹, and 1714 cm⁻¹.

16. A therapeutically effective amount of the polymorph of claim 14.

17. A pharmaceutical composition comprising the polymorph of claim 1.

18. The pharmaceutical composition of claim 17, wherein the polymorph is present in a therapeutically effective amount, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

19. A pharmaceutical composition comprising the polymorph of claim 6.

20. The pharmaceutical composition of claim 19, wherein the polymorph is present in a therapeutically effective amount, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

21. A pharmaceutical composition comprising the polymorph of claim 9.

22. The pharmaceutical composition of claim 21, wherein the polymorph is present in a therapeutically effective amount, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

23. A pharmaceutical composition comprising the polymorph of claim 14.

24. The pharmaceutical composition of claim 23, wherein the polymorph is present in a therapeutically effective amount, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

25. A method for producing a polymorph of 3-biphenyl-4-yl-(2S)-[(4′-trifluoromethyl-biphenyl-4-carbonyl)-amino]-propionic acid comprising: dissolving 3-biphenyl-4-yl-(2S)-[(4′-trifluoromethyl-biphenyl-4-carbonyl)-amino]-propionic acid in a solvent system comprising a solvent selected from the group consisting of: an alcoholic solvent, acetone, ethyl acetate, THF, HPCD, DMA, water, and mixtures thereof, and recovering the precipitate from the solvent system.

26. The method of claim 25, wherein the polymorph comprises the polymorph of claim 1.

27. The method of claim 25, wherein the polymorph comprises the polymorph of claim 6.

28. The method of claim 25, wherein the polymorph comprises the polymorph of claim 9.

29. The method of claim 25, wherein the polymorph comprises the polymorph of claim 14.

30. A method for treating thrombotic disorders comprising administering to a subject in need thereof the polymorph of claim 1.

31. The method of claim 30, wherein the polymorph is administered to the subject as a pharmaceutical composition comprising a therapeutically effective amount of the polymorph, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

32. A method for treating thrombotic disorders comprising administering to a subject in need thereof the polymorph of claim 6.

33. The method of claim 32, wherein the polymorph is administered to the subject as a pharmaceutical composition comprising a therapeutically effective amount of the polymorph, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

34. A method for treating thrombotic disorders comprising administering to a subject in need thereof the polymorph of claim 9.

35. The method of claim 34, wherein the polymorph is administered to the subject as a pharmaceutical composition comprising a therapeutically effective amount of the polymorph, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

36. A method for treating thrombotic disorders comprising administering to a subject in need thereof the polymorph of claim 14.

37. The method of claim 36, wherein the polymorph is administered to the subject as a pharmaceutical composition comprising a therapeutically effective amount of the polymorph, wherein a therapeutically effective amount is an amount sufficient to maintain in a subject a sustained blood level of 3-biphenyl-4-yl-(2S)-[(4′-trifluoromethyl-biphenyl-4-carbonyl)-amino]-propionic of greater than 0.1 μM.

38. A polymorph of 3-biphenyl-4-yl-(2S)-[(4′-trifluoromethyl-biphenyl-4-carbonyl)-amino]-propionic acid, Form III, having the following characteristics:
(a) having at least two X-ray powder diffractions peaks expressed in degrees-2θ selected from 15.7, 22.3, and 22.7; and
(b) a differential scanning calorimetry thermogram comprising an endothermic peak at about 294° C.

39. The polymorph of claim 38, further having one or more X-ray powder diffraction peaks expressed in degrees-2θ at about 3.8, 19.4, and 21.4.

40. The polymorph of claim 38, having no exothermic peaks.

41. A therapeutically effective amount of the polymorph of claim 38.

42. A pharmaceutical composition comprising the polymorph of claim 38.
